(19) United States
(12) Patent Application Publication
(10) Pub. No.: US 2008/0304621 A1
(43) Pub. Date: Dec. 11, 2008

(54) Display System For the Evaluation of Mammographies

(75) Inventor: Wolfhard Huber, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/573,578

(22) PCT Filed: Aug. 17, 2005
(86) PCT No.: PCT/IB05/52711

§ 371 Date: Feb. 12, 2007

(30) Foreign Application Priority Data
Aug. 18, 2004 (EP) 04300545.3

(51) Int. Cl. G09G 5/00; A61B 6/04

(52) U.S. Cl. 378/37; 345/619

(57) ABSTRACT

The invention relates to a display system that is particularly suited for a comparative evaluation of mammographies (L, R) of a patient which are displayed as mirror images in two display areas (2a, 2b). According to one aspect of the invention, a user may move a virtual magnifying glass (10a) in one of the images (L), and the system automatically derives the corresponding object related movement of a virtual magnifying glass (10b) in the other image (R). Thus the magnifying glasses (10a, 10b, 10a′, 10b′ are moved analogously with respect to the anatomy across the images. According to an alternative method, zoom windows always remain at the same position of the display area while the images under them are moved synchronously in an object related way.

### FIELD OF THE INVENTION

The invention relates to a display system for the comparative evaluation of images, particularly mammographies, an examination apparatus with such a display system, a method for the comparative evaluation of images, and a record carrier with software for carrying out said method.

### BACKGROUND OF THE INVENTION

In many applications it is necessary to compare images of one object in different states, for example images taken at different times, or to compare images of individually different but related objects. The following description will focus in this respect on medical applications, particularly on the evaluation of mammographies of the left and right breast of a patient, though the invention is not restricted to this special case.

In display systems that are currently used for the comparative evaluation of mammographies, sectional X-ray images of the left and right breast of a patient are displayed back to back as mirror images on neighboring display areas of two monitors. A physician can then see both images simultaneously and compare them in order to detect irregularities more easily. Moreover, it is known to provide a virtual “magnifying glass”, i.e. a window which can be moved across the screen steered by inputs with a keyboard or a mouse and in which the part of the image under the window is displayed in a magnified way. In order to compare related sub-regions in the two images, the physician has however to position separate magnifying glasses manually in the two images at the same anatomical location.

The US 2003/0063788 A1 discloses a display system for the correct registration of two images, e.g. a CT and a MR image, in which the whole images are first displayed side by side. A user may choose on the whole images reference points corresponding to the same object points, and the display of these reference points is then magnified in order to allow a fine adjustment of their position.

### SUMMARY OF THE INVENTION

Based on this situation it was an object of the present invention to provide means for a user-friendly comparative evaluation of images of related objects, particularly for the evaluation of mammographies.

This object is achieved by a display system according to claim 1, an examination apparatus according to claim 8, a method according to claim 9, and a record carrier according to claim 10. Preferred embodiments are disclosed in the dependent claims.

The display system according to the present invention allows the comparative evaluation of images of related objects, for example of mammographies of the left and right breast of a patient, and comprises the following components:

(i) Two or more display areas on which images may be displayed for a user. Typically, the display areas are realized by different monitors or by different windows on one monitor that do not overlap each other. The display areas may have different or preferably the same size.
(ii) An input device for entering a reference movement by a user, wherein the movement is defined in one of the aforementioned display areas. This display area may be predetermined and fixed for the display system, or it may be arbitrarily selectable by the user. The input device may for example be a conventional mouse and/or a keyboard with cursor keys. The reference movement may be expressed by screen coordinates, wherein a first pair: of screen coordinates describes the starting point and a second pair of screen coordinates describes the goal of the movement. The reference movement typically describes the shift of a focus point that is of interest for the user.
(iii) A control unit, for example a conventional data processing system (computer), that is adapted to perform the following steps:

a) The displaying of the images of the related objects in the aforementioned display areas, wherein each image is displayed in just one of the display areas.

b) The displaying of zoomed views of sub-regions of the images in the corresponding display areas. While the zooming may in principle be both a magnification (zoom-in) as well as a reduction (zoom-out), the typical application implies a magnified display of a sub-region of the image in order to allow a user are more detailed inspection thereof. Moreover, various image processing steps (e.g. contrast enhancement) may optionally be applied to the zoomed view. In the latter case, the zoom factor may particularly be one (i.e. identical size) and the special feature of the “zoomed view” is just the different processing.

c) The derivation of desired movements in each display area, wherein the derivation is based on a given reference movement in one selected display area which was entered by a user via the aforementioned input device, and wherein the derived desired movements are at least approximately identical with respect to the objects that are displayed in the corresponding display areas. If for example the entered reference movement shifts a certain point of the image in the selected display area from a location A to a location B, the desired movements in the other display areas are derived in such a way that they similarly move the corresponding object points A′ to corresponding object points B′. For the realization of this feature it is important that the displayed objects are related, i.e. that they all have corresponding object points (e.g. anatomical marks in medical applications).

d) The movement of the displayed images or alternatively of the zoomed views of the sub-regions according to the determined desired movements.

The described display system has the advantage that related objects like mammographies of the left and right breast are displayed simultaneously and that the movement of a focus point in one of said images is automatically transferred into object related movements in all images. In contrast to this, conventional display systems for mammographies move images in all display areas in the same direction of the screen (e.g. downward), which may be disturbing for the physician and require an additional repositioning. Such problems are avoided with the proposed display system because of the object orientation of the desired movements in all display areas.

According to a preferred embodiment of the display system, the zoomed views of the sub-regions are displayed as inserts in the corresponding images just above the viewed sub-regions. The views then behave in an intuitive way like magnifying glasses which are moved relative to an image and which magnify the sub-region below the glass.

The related objects may particularly be displayed as mirror images in the different display areas. Such a mirrored display is for example applied in usual reporting stations for mammographies, wherein the left and right breasts of a patient are displayed back to back. If it is known that such a mirrored display is used, then the derivation of desired movements from a reference movement may be simplified to just a mirroring of the reference movement.

As already mentioned, special image processing procedures (e.g. contrast enhancement) may be performed for the zoomed views in order to allow an optimal representation of regions of interest. Moreover, display parameters of the zoomed views may preferably be interactively set by a user. Such user defined display parameters may particularly comprise the magnification of the zoomed region or parameters of the aforementioned special image processing procedures.

The invention further comprises an examination apparatus with

an imaging system for generating images of different objects, particularly an X-ray device for generating mammographies of the breasts of a patient;

a display system of the kind described above.

Moreover, the invention relates to a method for the comparative evaluation of images of related objects, comprising the following steps:

a) displaying said images in different display areas;
b) displaying zoomed views of sub-regions of said images in the display areas;
c) deriving desired movements in each display area from a given reference movement in one display area such that said desired movements are at least approximately identical with respect to the corresponding objects that are displayed in the display areas;
d) movement of the displayed images or of the zoomed views according to the desired movements.

Finally, the invention comprises a record carrier, for example a floppy disk, a hard disk, or a compact disc (CD), on which a computer program for the comparative evaluation of images of related objects is stored, wherein said program is adapted to execute a method of the aforementioned kind.

The examination apparatus, the method, and the record carrier have similar features like a display system as it was described above. For more information on details, advantages and further developments of them reference is therefore made to the description of said display system.

### BRIEF DESCRIPTION OF THE DRAWINGS

In the following the invention is described by way of example with the help of the accompanying drawings in which:

FIG. 1 schematically shows a display system according to the present invention that is used for the comparative evaluation of mammographies;

FIG. 2 shows the movement of magnifying glasses across display areas according to the present invention;

FIG. 3 shows the movement of displayed images under a zoom window according to the present invention.

### DESCRIPTION OF PREFERRED EMBODIMENTS

A preferred embodiment of the invention will in the following be described with respect to the example of a comparative evaluation of mammographies in a review/reporting station, though the invention is not restricted to such an application.

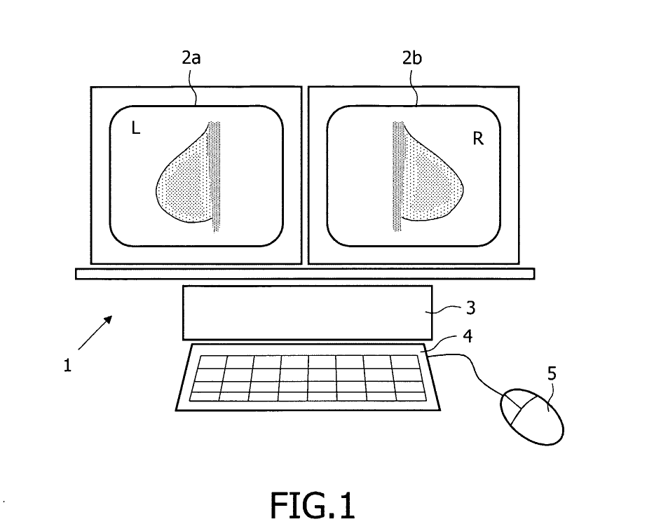

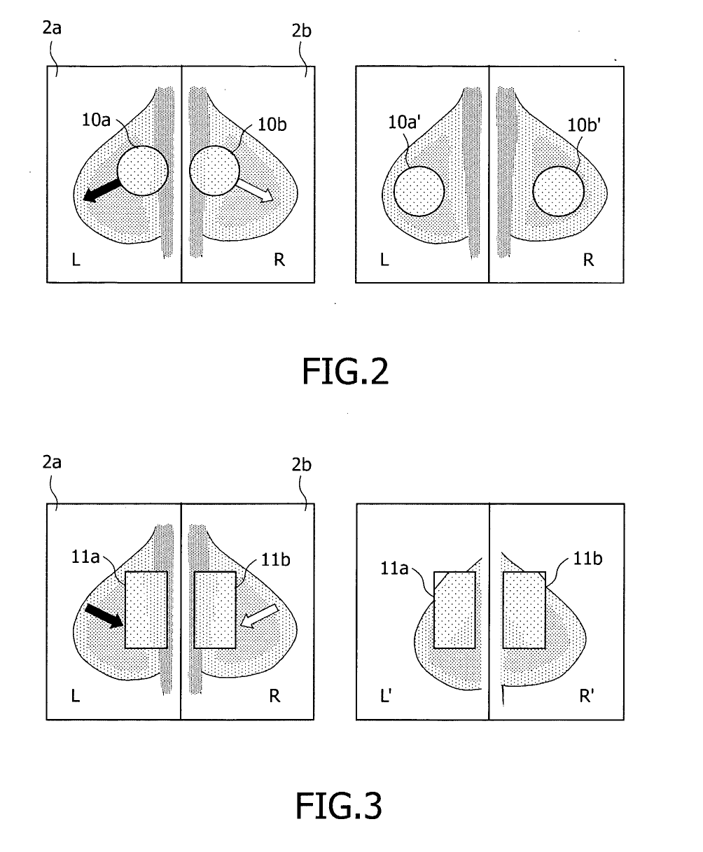

FIG. 1 shows a typical reporting station or display system 1 according to the present invention which comprises a control unit 3 in the form of a computer (workstation) with typical components like central processing unit, memory, I/O interfaces, etc. and appropriate software. Connected to the control unit 3 are two high resolution monitors with two display areas 2a and 2b of identical size and input devices like a keyboard 4 and a mouse 5. According to the conventions in the diagnosis of mammographies, a sectional image L of the left breast and a sectional image R of the right breast of a patient are displayed in a mirrored way back to back in the two display areas 2a, 2b. Typically a third monitor or a third display area (not shown) is provided for the display of patient data. Moreover, the display areas could alternatively be realized as different windows on one monitor (cf. FIGS. 2, 3).

For making a diagnosis on the displayed mammographies, a physician has to compare the two images L, R and inspect suspicious sub-regions in more detail if necessary. In order to facilitate such a diagnosis, the display system 1 is adapted to provide the functions described in the following with respect to FIGS. 2 and 3 which only depict the corresponding display areas 2a, 2b back to back.

A first technique provided by the display system 1 is shown in FIG. 2. In this case, (virtual) magnifying glasses 10a, 10b are provided in the corresponding images L, R, wherein the sub-region of the images that lies under the magnifying glasses 10a, 10b is displayed magnified in the insert corresponding to the magnifying glasses. (The magnifying glasses are depicted in FIG. 2 as circles, though in practice they will preferably be rectangular.)

A user who wants to inspect another sub-region of the mammographies may select one of the magnifying glasses, for example the left one 10a, and move it across the corresponding display area 2a (for example by dragging it with a mouse 5 or by inputs with cursor keys). A typical reference movement entered by a user is indicated by a black arrow in the left part of FIG. 2. If a synchronization mode is activated, the display system 1 then calculates a corresponding, object related desired movement of the magnifying glass 10b in the other image R (white arrow). Because the breasts are displayed as mirror images and the movement of the magnifying glasses shall be object related, the movement of the magnifying glass 10b will be mirrored with respect to the reference movement of the magnifying glass 10a. The right part of FIG. 2, which depicts the position of the magnifying glasses 10a′, 10b′ after the execution of the desired movements, reveals that the final positions of the magnifying glasses 10a′, 10b′ are (approximately) identical with respect to the anatomy of the displayed objects. Thus a physician may immediately inspect and compare corresponding anatomical sub-regions of the left and right images L, R.

FIG. 3 shows another optional display mode provided by the display system 1. In this case, zoom windows 11a, 11b are provided in the display areas 2a, 2b at predetermined and fixed positions, for example near the centre of the display areas. As in the case of the magnifying glasses of FIG. 2, the sub-region of the images L, R that lies under the zoom windows 11a, 11b is displayed in the corresponding window as a magnified view (zoomed-in). The magnification factor may be selected arbitrarily by a user and may also comprise magnifications smaller than 1 (i.e. a zooming-out) though this case is of less practical relevance.

A user who wants to inspect a different sub-region of the displayed object may again choose one of the display areas 2a, 2b and enter a desired reference movement of the image displayed there. In the example of FIG. 3, the user selects for example the left image L and indicates by a mouse or a keyboard a downward reference movement to the right of that image (black arrow). The imaging system then calculates a corresponding object-related movement of the right image R (white arrow) which is in the present example a mirroring of the reference movement entered by the user.

The right part of FIG. 3 shows the display areas after the execution of the image movements. In this case, the zoom windows 11a, 11b remain at their positions, while the images under them move according to the reference movement and the derived desired movement, yielding shifted images L′, R′. The contents in the zoom windows 11a, 11b changes accordingly to represent the sub-regions of the images L′ and R′ under these windows.

A great advantage of the method of FIG. 3 is that the physician may concentrate on the same area of the screen, i.e. the zoom windows 11a, 11b, which causes less fatigue than the tracking of zoom windows which move across the screen. The methods provides at the same time a good orientation with respect to the whole objects because the zoom windows are always displayed above the magnified sub-region.

The described method of moving a magnifying glass 10a, 10b or an image L, R may readily be generalized to cases in which the displayed images show an object not only in a mirrored way but with a more complex transformation (for example a shift, a rotation, a rescaling or the like). In this case, a reference movement entered by a user must be transformed accordingly (i.e. shifted, rotated, rescaled or the like) in order to guarantee that the focus is moved in all images in an object related way.

The image parts displayed in the magnifying glasses 10a, 10b or in the zoom windows 11a, 11b may also be subject to a special image processing, e.g. a color coding, a contrast enhancement or the like, in which cases zooming may be omitted (zoom factor 1.0).

Finally it is pointed out that in the present application the term “comprising” does not exclude other elements or steps, that “a” or “an” does not exclude a plurality, and that a single processor or other unit may fulfill the functions of several means. Moreover, reference signs in the claims shall not be construed as limiting their scope.

1. Display system (1) for the comparative evaluation of images (L, R, L′, R′) of related objects, comprising
at least two display areas (2a, 2b);
an input device (4, 5) for entering a reference movement in a selected one of the display areas (2a);
a control unit (3) that is adapted to perform the following steps:
a) displaying said images (L, R, L′, R′) of the related objects in the display areas (2a, 2b);
b) displaying zoomed views (10a, 10b, 10a′, 10b′, 11a, 11b) of sub-regions of said images (L, R, L′, R′) in the display areas (2a, 2b);
c) deriving desired movements in each display area (2a, 2b) from a given reference movement in one display area (2a) such that said desired movements are at least approximately identical with respect to the corresponding objects that are displayed in the display areas (2a, 2b);
d) movement of the displayed images (L, R, L′, R′) or of the zoomed views (10a, 10b, 10a′, 10b′) according to the desired movements.

2. The display system (1) according to claim 1, characterized in that the zoomed views (10a, 10b, 10a′, 10b′, 11a, 11b) of the sub-regions are displayed as inserts in the corresponding images (L, R, L′, R′) at positions corresponding to said sub-regions.

3. The display system (1) according to claim 1, characterized in that the images (L, R, L′, R′) of the related objects are medical images of the left and right breasts of a patient, particularly X-ray images.

4. The display system (1) according to claim 1, characterized in that the objects are displayed as mirror images in two different display areas (2a, 2b).

5. The display system (1) according to claim 1, characterized in that the display areas (2a, 2b) have the same size.

6. The display system (1) according to claim 1, characterized in that the input device comprises a mouse (5) and/or a keyboard (4).

7. The display system (1) according to claim 1, characterized in that special image processing procedures are performed for the zoomed views (10a, 10b, 10a′, 10b′, 11a, 11b), the parameters of which may particularly be interactively set.

8. Examination apparatus, comprising
an imaging system for generating images of different objects, particularly an X-ray device for mammography;
a display system according to claim 1.

9. A method for the comparative evaluation of images (L, R, L′, R′) of related objects, comprising the following steps:
a) displaying said images (L, R, L′, R′) in different display areas (2a, 2b);
b) displaying zoomed views (10a, 10b, 10a′, 10b′, 11a, 11b) of sub-regions of said images (L, R, L′, R′) in the display areas (2a, 2b);
c) deriving desired movements in each display area (2a, 2b) from a given reference movement in one display area (2a) such that said desired movements are at least approximately identical with respect to the corresponding objects that are displayed in the display areas (2a, 2b);
d) movement of the displayed images (L, R, L′, R′) or of the zoomed views (10a, 10b, 10a′, 10b′) according to the desired movements.

10. A record carrier on which a computer program for the comparative evaluation of digital images is stored, said program being adapted to execute a method according to claim 9.
